(19) United States
(12) Patent Application Publication
(10) Pub. No.: US 2018/0221297 A1
(43) Pub. Date: Aug. 9, 2018

(54) System and Method for Improving Adhesion of Transdermal Delivery Devices

(71) Applicant: Kirti H. Valia, Plainsboro, NJ (US)

(72) Inventor: Kirti H. Valia, Plainsboro, NJ (US)

(21) Appl. No.: 15/887,345

(22) Filed: Feb. 2, 2018

Related U.S. Application Data
(60) Provisional application No. 62/454,203, filed on Feb. 3, 2017.

(51) Int. Cl. A61K 9/70; A61K 9/00; A61K 31/4468; A61K 31/4168; B32B 38/10; B32B 37/12; B32B 37/10; B32B 37/18; B32B 37/20; B32B 7/12; B32B 27/08; B32B 27/30; B32B 27/36

(52) U.S. Cl. CPC A61K 9/7038; B32B 2556/00; A61K 9/7084; A61K 31/4468; A61K 31/4168; B32B 38/10; B32B 37/1207; B32B 37/10; B32B 37/182; B32B 37/203; B32B 7/12; B32B 27/08; B32B 27/308; B32B 27/36; B32B 27/306; B32B 2305/72; B32B 2333/00; B32B 2367/00; B32B 2331/04; A61K 9/0014

(57) ABSTRACT

Transdermal drug delivery devices and systems with improved adhesion are disclosed. The transdermal drug delivery devices, or patches, include an integrated overlay that provides a drug-free adhesive perimeter designed to avoid affecting delivery of drug from the device. Also disclosed are methods of manufacturing the transdermal drug delivery devices that include incorporating an integrated overlay onto a TDS patch.

### CROSS-REFERENCE TO RELATED APPLICATIONS

This claims benefit of U.S. Provisional Application Nos. 62/454,203, filed Feb. 3, 2017, the entire contents of which are incorporated by reference herein.

### FIELD

The invention relates to devices, systems, and methods of improving the adhesion of transdermal drug delivery patches.

### BACKGROUND

Transdermal drug delivery systems (TDS), such as TDS patches, offer advantages over more conventional oral or parenteral dosage forms. First, the administration of the drug is non-invasive, and drug delivery can be sustained for several days or more from a single patch, thereby eliminating the need for repeated oral dosage. In addition, the delivery of the drug can be controlled without peaks and valleys, resulting in better side effect profiles, effectiveness, and compliance. However, the usefulness of the TDS patch relies, in part, on the adhesion of the patch to the skin of an individual.

The drug release rate of a TDS patch is controlled by a variety of factors. The chemical composition of the TDS patch, including the various polymeric films and adhesives can affect the drug's pharmacokinetics (PK). In addition, the release rate and dosage of the drug from the TDS patch to the skin can be influenced by the surface area of the adhesive. Indeed, poor contact, such as lifting or peeling of the patches during the period of application, can reduce the dosage of drug delivered to the individual. Traditional means of improving patch adhesion during wear include instructing the patient to press down on the patch to improve adhesion or placing another patch or similar device, called an overlay, over the TDS patch in order to keep it from falling off. However, these methods are inefficient as continually pressing on the TDS patch does not overcome the lack of sufficient contact over time, and the use of separate overlays requires careful alignment of the overlay over the TDS patch.

In addition, proposed guidance by the Federal Drug Administration has highlighted an emphasis on the adhesive properties of TDS patches that require the industry to test TDS patches using an adhesive scale and ranking system, to use reasonable efforts to optimize adhesive, and take measures to ensure that no increase in skin sensitivity or irritation results due to modifications done to meet the adhesion requirements. See U.S. Dept. of Health and Human Srvs, F.D.A. CDER, “Assessing Adhesion with Transdermal Delivery Systems and Topical Patches for ANDAs-Draft Guidance for Industry” (June 2016). However, simply altering the adhesive formulations may impact the PK of the TDS patch. Thus, these potential guidelines may provide even greater burdens for the manufacturer, and subsequently generic drug makers that manufacture drugs and medical devices that are equivalent to a reference listed drug (RLD).

Thus, there exists a need for transdermal drug delivery devices with improved adhesion that reduce or eliminate peeling or lifting without changing the release rate or other pharmacokinetics of the drug or pharmaceutical agent contained within the device.

### SUMMARY

Aspects of the present invention features a transdermal drug delivery system (TDS), such as a patch, with an integrated overlay that improves the adhesion of the device. In addition, provided herein are methods for improving the adhesion of a TDS patch by fabricating a suitable overlay and integrating that overlay onto the patch.

One aspect of the invention features a method of increasing the adhesion of a transdermal drug delivery patch and includes the steps of (a) providing a transdermal drug delivery patch that includes a patch adhesive layer (that includes a first elastomer) and a patch backing layer; (b) fabricating an overlay by coating an overlay adhesive layer on an overlay release liner and laminating an overlay backing layer onto the overlay adhesive layer (that includes a second elastomer which is substantially the same elastomer as the first elastomer), and where the overlay adhesive layer has a surface area that is greater than a surface area of the patch adhesive layer; (c) removing the overlay release liner whereby the overlay adhesive layer is exposed; and (d) integrating the transdermal delivery patch and the overlay by disposing the overlay on the patch backing layer so that the overlay adhesive layer contacts the patch backing layer to produce a transdermal drug delivery patch with integrated overlay having an extended adhesive surface area. In this aspect, the transdermal drug delivery patch with integrated overlay has an increased peel adhesion as compared to the transdermal drug delivery patch.

In certain embodiments, the patch backing layer has a first polymer composition, and the overlay backing layer has a second polymer composition, provided that the first polymer composition and second polymer composition include substantially the same polymers. In yet other embodiments, the overlay is adhered to the transdermal drug delivery patch by heat curing.

In some embodiments, the transdermal drug delivery patch with integrated overlay has a peel adhesion that is at least 1.5-fold greater as compared to the transdermal drug delivery patch. In other embodiments, it has a peel adhesion that is at least 2-fold greater as compared to the transdermal drug delivery patch. In yet other embodiments, the overlay adhesive layer has a thickness of at least 1 mil. In still other embodiments, the overlay adhesive layer has a thickness of at least 2 mil.

In some embodiments of the methods, the first elastomer is selected from the group consisting of polyacrylate, polyisobutylene (PIB), natural rubber, silicone, and styrene rubber. In other embodiments, the method includes a step done prior to coating the overlay adhesive layer onto the overlay backing layer, which includes increasing the peel adhesion of the overlay adhesive layer as compared to the patch adhesive layer. In certain embodiments, increasing the peel adhesion of the overlay adhesive layer includes adding a tackifying resin to the overlay adhesive layer. In other embodiments, the first elastomer is PI 3, and the method includes increasing the peel adhesion of the overlay adhesive layer by altering the ratio of high molecular weight PIB to low molecular weight PIB in the overlay adhesive layer. In yet other embodiments, both the first elastomer and the second elastomer are polyacrylates comprising hydroxyl functional groups.

In some embodiments, the transdermal drug delivery patch is a reservoir-type transdermal patch or a drug-in adhesive transdermal patch. In a particular embodiment, the transdermal delivery patch is a drug-in adhesive transdermal patch, and the patch adhesive layer comprises a therapeutically effective amount of a drug. Alternatively, the transdermal drug delivery patch is a reservoir-type transdermal patch comprising a drug reservoir, and the drug reservoir comprises a therapeutically effective amount of a drug. In either of these embodiments, the drug is selected from the group consisting of clonidine, scopolamine, oxybutynin, lesopritron, estradiol, levonorgestrel, fentanyl, albuterol, labetalol, atropine, haloperidol, isosorbide dinitrate, nitroglycerin, norethindrone acetate, nicotine, benztropine, secoverine, dexsecoverin, arecoline, buprenorphine, donepezil hydrochloride, donepezil base, lidocaine, selegiline, rivastigmine, methylphenidate, diclofenac, ondansetron, varencicline, oxymorphone, rotigotine, and granisetron.

In some embodiments, the transdermal drug delivery patch with integrated overlay includes a patch release liner disposed on the patch adhesive layer whereby the patch release liner covers the extended adhesive surface area. In other embodiments, the method includes removing the patch release liner and contacting the patch adhesive layer of the transdermal drug delivery patch with integrated overlay to skin of an individual.

Another aspect of the invention features a transdermal drug delivery patch with integrated overlay produced as summarized above. In other aspects, the invention features an overlay produced as summarized above.

Another aspect of the invention features a transdermal drug delivery patch with integrated overlay that includes (a) a transdermal drug delivery patch with a patch adhesive layer and a patch backing layer, wherein the patch adhesive layer comprises a first elastomer and the patch backing layer comprises a first polymer composition; and (b) an overlay disposed on the patch backing layer, the overlay comprising an overlay backing layer and an overlay adhesive layer, wherein the overlay adhesive layer comprises a second elastomer that is substantially the same elastomer as the first elastomer. In this aspect, the overlay adhesive layer has a surface area that is greater than a surface area of the patch adhesive layer, and the overlay adhesive layer is attached to the patch backing layer. Also in this aspect, the transdermal drug delivery patch with integrated overlay has an extended adhesive surface area and an increased peel adhesion as compared to the transdermal drug delivery patch.

In some embodiments, the patch backing layer has a first polymer composition, and the overlay backing layer has a second polymer composition, provided that the first polymer composition and second polymer composition comprise substantially the same polymers. In a particular embodiment, the first elastomer is selected from the group consisting of polyacrylates, polyisobutylene (PIB), natural rubber, silicone, and styrene rubber.

In some embodiments, the overlay adhesive layer further comprises a tackifying resin. In other embodiments, the first elastomer is PIB, and the second elastomer is PIB having an different ratio of high molecular weight PIB to low molecular weight PIB as compared to the first elastomer. Alternatively, both the first elastomer and the second elastomer are polyacrylates comprising hydroxyl functional groups.

In some embodiments, the transdermal drug delivery patch with integrated overlay includes a patch release liner disposed on the surface of the patch adhesive layer whereby the patch release liner covers the extended adhesive surface area. In other embodiments, both the first polymer composition and the second polymer composition comprise one or more synthetic polymers selected from the group consisting of polyolefin oils, polyester, polyethylene, polyvinylidine, chloride, and polyurethane.

Another aspect of the invention features a method of transdermally administering a therapeutically effective amount of a drug to an individual in need of such drug that includes the steps of providing a transdermal drug delivery patch with integrated overlay as summarized above and applying it to the intact skin of the individual for a minimum amount of time, e.g., at least about 2 days.

In some embodiments, the transdermal drug delivery patch with integrated overlay is applied to the intact skin of the individual for at least about 4 days. In other embodiments, the transdermal drug delivery patch with integrated overlay is applied to the intact skin of the individual for at least about 7 days.

### BRIEF DESCRIPTION OF THE FIGURES

FIG. 1A is a cross-sectional depiction of an embodiment of a reservoir-type transdermal drug delivery patch.

FIG. 1B is a cross-sectional depiction of an embodiment of a drug-in adhesive transdermal drug delivery patch.

FIG. 1C is a cross-sectional depiction of an embodiment of an overlay of the present invention.

FIG. 2 is a chart providing a non-limiting list of pressure-sensitive adhesive compositions and the corresponding performance properties (peel adhesion, dynamic shear, and tack). The chart is adapted from the “DURO-TAK® Transdermal Grade Pressure Sensitive Adhesives” chart that can be found in its entirety on the National Adhesives website.

FIG. 3A is a diagram depicting cross-sectional views of an exemplary fabrication process for an embodiment of a transdermal drug delivery patch with integrated overlay. The circled numbers represent different stages of the fabrication process.

FIG. 3B is a continuation of FIG. 2A and depicts an exemplary fabrication process for an embodiment of a transdermal drug delivery patch with integrated overlay. The circled numbers represent different stages of the fabrication process.

FIG. 4A is a cross-sectional depiction of an embodiment of a transdermal drug delivery patch with integrated overlay of the present invention.

FIG. 4B is a cross-sectional depiction of an embodiment of a transdermal drug delivery patch with integrated overlay of the present invention.

FIG. 4C depicts a bottom view of an embodiment of a transdermal drug delivery patch with integrated overlay of the present invention.

FIG. 4D depicts a bottom view of an embodiment of a transdermal drug delivery patch with integrated overlay of the present invention.

FIG. 5A is a picture of the adhesive layer of an embodiment of a transdermal drug delivery patch with integrated overlay of the present invention.

FIG. 5B is a picture of the adhesive layer of an embodiment of a transdermal drug delivery patch with integrated overlay of the present invention.

### DETAILED DESCRIPTION

All percentages expressed herein are by weight of the total weight of the composition or mixture unless expressed otherwise. All ratios expressed herein are on a weight (w/w) basis unless expressed otherwise.

Ranges may be used herein in shorthand, to avoid having to list and describe each value within the range. Any appropriate value within the range can be selected, where appropriate, as the upper value, lower value, or the terminus of the range.

As used herein, the singular form of a word includes the plural, and vice versa, unless the context clearly dictates otherwise. Thus, the references “a”, “an”, and “the” are generally inclusive of the plurals of the respective terms. For example, reference to “a method” or “an adhesive” includes a plurality of such “methods”, or “adhesives.” Likewise the terms “include”, “including”, and “or” should all be construed to be inclusive, unless such a construction is clearly prohibited from the context. Similarly, the term “examples,” particularly when followed by a listing of terms, is merely exemplary and illustrative and should not be deemed exclusive or comprehensive.

The term “comprising” is intended to include embodiments encompassed by the terms “consisting essentially of” and “consisting of”. Similarly, the term “consisting essentially of” is intended to include embodiments encompassed by the term “consisting of”

The methods and compositions and other advances disclosed herein are not limited to particular equipment or processes described herein because such equipment or processes may vary. Further, the terminology used herein is for describing particular embodiments only and is not intended to limit the scope of that which is disclosed or claimed.

Unless defined otherwise, all technical and scientific terms, terms of art, and acronyms used herein have the meanings commonly understood by one of ordinary skill in the art in the field(s) of the invention, or in the field(s) where the term is used. Although any compositions, methods, articles of manufacture, or other means or materials similar or equivalent to those described herein can be used in the practice of the present invention, the preferred compositions, methods, articles of manufacture, or other means or materials are described herein.

The term “about” refers to the variation in the numerical value of a measurement, e.g., temperature, viscosity average molecular weight, width, thickness, weight percentage, etc., due to typical error rates of the device used to obtain that measure. In one embodiment, the term “about” means within 5% of the reported numerical value.

The term “drug” or “pharmaceutical agent” as used herein refers to any chemical or biological material or compound suitable for transdermal administration.

The term “elastomer” as used herein means a natural or synthetic polymer with viscoelasticity (i.e., having both viscosity and elasticity) and having weak inter-molecular forces, a low Young's modulus, and high failure strain as compared to other materials. The term “elastomer” may refer to a type or class of elastomer (e.g., acrylates and polyacrylates, silicones, polyisobutylenes, natural rubbers, styrene rubbers) or a specific elastomer composition comprising polymers of the same elastomer type or class (e.g., a 1:2 ratio of high molecular weight polyisobutylenes (viscosity average molecular weight of about 1,250,000) to low molecular weight polyisobutylenes (viscosity average molecular weight of about 45,000). Thus, reference to “substantially the same elastomer” refers to two or more elastomer compositions comprising elastomers of the same type or class, but may have different viscosities, peel adhesions properties, tack, and the like.

The term “therapeutically effective amount” as used herein refers to an amount of drug or pharmaceutical agent that is nontoxic but sufficient to provide the desired local or systemic effect.

The term “pressure sensitive adhesive” or “PSA” as used herein is an adhesive that includes at least one elastomer and is a component in an adhesive layer that enables, e.g., a TDS patch to adhere to a surface at room temperature by temporary application of pressure alone.

The term “release rate” as used herein refers to the rate at which the drug or pharmaceutical agent is released from a TDS device of the disclosure to a surface on which the transdermal delivery system or device is applied.

The term “viscosity average molecular weight” refers to the average molecular weight of a polymer obtained by measuring the viscosity according to the Mark-Houwink equation.

The terms “transdermal” or “transdermal delivery” or “transdermally” as used herein relate to or denote the application of a drug or pharmaceutical agent through the skin, typically by using an adhesive patch, so that it is absorbed slowly into the body.

The terms “transdermal delivery device”, “transdermal delivery system”, “transdermal delivery patch”, “TDS patch”, and “TDS device” refer to devices or patches, and include topical patches, that are applied to the intact skin of an individual for transdermal delivery of a drug. All patents, patent applications, publications, technical and/or scholarly articles, and other references cited or referred to herein are in their entirety incorporated herein by reference to the extent allowed by law. The discussion of those references is intended merely to summarize the assertions made therein. No admission is made that any such patents, patent applications, publications or references, or any portion thereof, are relevant, material, or prior art. The right to challenge the accuracy and pertinence of any assertion of such patents, patent applications, publications, and other references as relevant, material, or prior art is specifically reserved.

In one aspect, the invention features a TDS device or patch, including, but not limited to, reservoir-type-membrane-controlled patches and drug-in-adhesive patches, with an integrated overlay that has improved peel adhesion compared to an equivalent TDS device or patch without the overlay. In some embodiments, the integrated overlay is attached or adhered to the backing layer of a TDS patch. In certain aspects, the integrated overlay includes a backing layer and an adhesive layer, and the adhesive layer of the overlay facilitates its attachment to the TDS patch. In these aspects, it is preferable that the overlay is longer and wider than the TDS patch such that the adhesive layer of the overlay provides a drug-free, adhesive-only border around the TDS patch thereby extended the total adhesive surface area of the TDS patch. In preferred aspects, the backing layer of the overlay and the backing layer of the TDS patch are made from the same polymers (e.g., both backing layers are films made from polyester). In particular embodiments, the adhesive layers of both the overlay and the TDS patch contain the same adhesives or contain the same elastomers or substantially the same elastomers (i.e., elastomers of the same type), including, but not limited to, acrylates or polyacrylates, silicone, polyisobutylenes (PIBs), styrene rubber, natural rubber, and the like. In a more preferred embodiment, the adhesive layer of the overlay contains an elastomer that is modified in some way (as compared to the TDS patch) to improve the peel adhesion of the overlay as compared to the TDS patch or, alternatively, the adhesive layer includes one or more additives (such as tackifying resins) that are present in addition to the elastomer to improve the peel adhesion of the overlay as compared to the patch. Also provided are methods for fabricating a TDS patch with integrated overlay that include attaching an overlay onto a TDS patch. In such methods, it is preferable to first fabricate the overlay such that it includes an adhesive layer containing an elastomer of the same type as the TDS patch. In more preferred aspects, the adhesive layer of the overlay is modified to have improved peel adhesion as compared to the adhesive layer of the TDS patch.

The various components of the TDS patch with integrated overlay will now be explained in more detail by way of non-limiting exemplary embodiments.

### The Transdermal Drug Delivery Device

Provided herein are TDS patches that enable a drug or pharmaceutical agent to be transdermally administered in therapeutically effective amounts to individuals by way of Preferably, a TDS patch is applied to an area of intact skin on a human patient of from about 5 cm² to about 200 cm² over an extended period of time, e.g., at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 4 days, 5 days, 6 days, 7 days, 8 days, or more.

Several factors affect the delivery rate or release rate of a drug contained in a TDS patch of the invention. Surface area and drug concentration are both considered when fabricating a TDS patch for delivering therapeutically effective amounts of drug to a patient. In general, a larger surface area of contact between the TDS patch and the skin of the individual provides a higher dosage of drug delivery. Further, it is known in the art that the release rate of the drug depends on the concentration gradient of the drug between the drug reservoir or drug-in adhesive and the skin of the patient, and may require a much higher concentration of the drug in the patch to enable transdermal drug delivery. In addition, depending on the drug to be delivered, the adhesive composition may affect the release rate. Finally, a TDS patch should be sufficiently attached to the intact skin of the patient, and peeling or loosening of the patch decreases the contact area between the TDS patch and the skin and can result in a decrease in the dosage of drug delivered to the patient. Therefore, it is an object of this invention to improve the adhesion of a TDS patch by integrating an overlay to increase the adhesive surface area and improve the adhesion of the TDS patch.

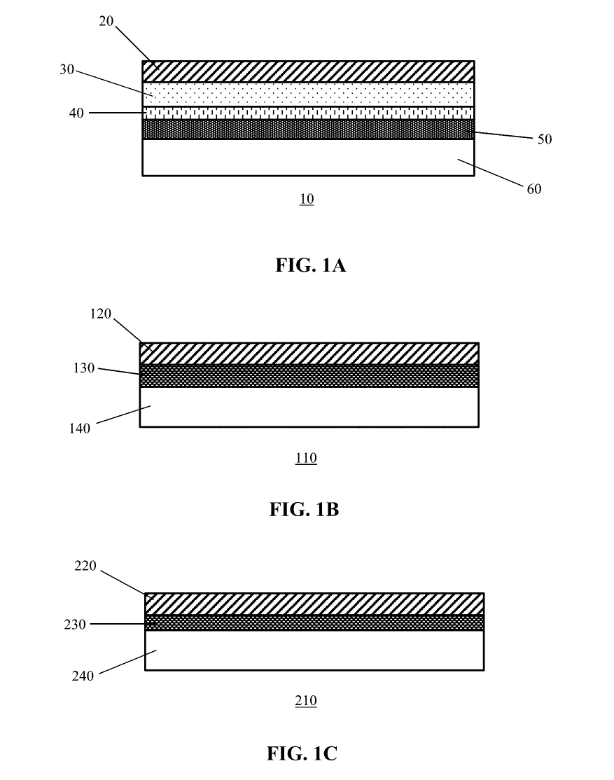

FIG. 1A depicts a reservoir-type TDS patch 10, which comprises a backing layer 20, a drug reservoir 30 containing the drug to be delivered, a rate-controlling membrane 40, and an adhesive layer 50 that includes a pressure sensitive adhesive (PSA). In a preferred embodiment, the reservoir-type TDS patch 10 includes a release liner 60. The drug reservoir 30 may include a mixture of drug and an adhesive or, alternatively, be in the form of a gel containing the drug. In preferred embodiments, the adhesive layer 50, which is the layer that is in substantially direct contact with the intact skin during application, also contains the drug to be delivered, albeit at a smaller concentration compared to the drug in the drug reservoir. Reservoir-type TDS patches are well known in the art as described in U.S. Pat. No. 9,248,104, U.S. Pat. No. 4,201,211, and U.S. Pat. No. 4,588,580, the entire contents of each of which are incorporated herein by reference, and some reservoir-type TDS patches are commercially available (e.g., CATAPRES-TTS clonidine TDS, Boehringer Ingelheim Pharmaceuticals, Inc., Ridgefield, Conn., USA).

FIG. 1B depicts a drug-in-adhesive TDS patch 110, which comprises a backing layer 120 and an adhesive layer 150 comprising both a matrix type adhesive and a drug to be administered transdermally to a patient. In a preferred embodiment, the drug-in-adhesive TDS patch 110 includes a release liner 140. Drug-in-adhesive TDS patches are well known in the art as described in U.S. Pat. No. 9,248,104, US 2014/0005617 A1, and U.S. Pat. No. 5,948,433, the entire contents of each of which are incorporated herein by reference, and some drug-in-adhesive TDS patches are commercially available (e.g., fentanyl TDS, Mylan Pharmaceuticals, Inc., Morgantown, W. Va., USA and DURAGESIC fentanyl TDS system, Janssen Pharmaceutical Products, LP, Titusville, N.J., USA)

The backing layer (also referred to as backing film or backing) serves as a support for some embodiments of the TDS patches suitable for use herein and is typically disposed on the upper surface of the TDS patch as it functions as the primary structural element and provides the TDS patch with its flexibility. In a reservoir-type TDS patch, it is preferable that the backing layer be disposed behind or on the surface of the drug reservoir opposite that of the adhesive layer (see FIG. 1A). In the drug-in-adhesive TDS patch, it is preferable that the backing layer be disposed on the adhesive layer (see FIG. 1B). Preferably, the backing layer is substantially impermeable to the drug contained in the TDS device. The backing layer is typically made of a sheet or film of a flexible elastomeric material and is preferably nonbreathable. Non-occlusive backing layers allow the area to breath (i.e., promote water vapor transmission from the skin surface), while occlusive backing layers reduce air/vapor permeation. Preferably, the backing layer is occlusive in the TDS patches shown in FIGS. 1A and 1B.

Backing layers or films for use in TDS patches of the present disclosure are usually derived from synthetic polymers like polyolefin oils, polyester, polyethylene, polyvinylidine chloride, and polyurethane and may be multilaminates comprising more than one layer of synthetic polymer. Typically, the thickness of the backing layer is from about 0.5 mil to about 5 mils; more particularly, the thickness is from about 1 mil to about 3 mils. In one embodiment, the backing layer is a 2.8 mil multilayer laminate of pigmented polyethylene, aluminum vapor coated polyester, and an ethylene vinyl acetate heat-seal layer (e.g., 3M SCOTCHPAK 9730, 3M Drug Delivery Systems, St. Paul, Minn., USA) or a 2 mil polyester and ethyl vinyl acetate (EVA) polymer (e.g., 3M SCOTCHPAK 9733, 3M Drug Delivery Systems, St. Paul, Minn., USA).

The rate-controlling membrane of the reservoir-type TDS patch shown in FIG. 1A permits controlled delivery of the drug or pharmaceutical agent from the drug reservoir into the adhesive layer. Rate-controlling membranes for use with patches of the invention are well known in the art and selection is readily accomplished by an ordinary practitioner. Suitable rate-controlling membranes include, but are not limited to, thin semi-permeable, ethylene vinyl acetate co-polymer membranes or thin microporous membranes of polyethylene, and polypropylene. Suitable rate-controlling membranes are commercially available, for example, from 3M Drug Delivery Systems, St. Paul, Minn., USA and Celgard, LLC, Charlotte, N.C. The rate-controlling membrane can be from about 0.5 mil to about 5 mils thick and preferably about 1 mil to about 3 mils thick.

In preferred aspects, a TDS patch includes an adhesive layer, which comprises a pressure-sensitive adhesive (PSA). PSAs contain elastomers, including synthetic polymers with viscoelasticity. PSAs for use with patches of the invention are well known in the art and selection is readily accomplished by an ordinary practitioner. In some aspects, it is desirable to determine one or more physical or performance characteristics of the adhesive layer in the TDS patch, such as peel adhesion, tack, and dynamic shear. Peel adhesion is a measure of the amount of force needed to separate the adhesive layer (e.g., a TDS patch) from a substrate. Peel adhesion can also be used to determine the overall adhesiveness of the adhesive layer. Tack is a common measure of the adhesive layers initial adhesion and release from a substrate. Dynamic shear is a test to determine the ability of an adhesive layer to stick to a substrate when subjected to dynamic forces or movement. Thus, the choice of PSA in the adhesive layer of a TDS patch may be based, among other things, on any one or more of these performance characteristics.

Preferably, the PSAs used in the TDS patches should have good cohesion and low dermal irritation. In an embodiment, the TDS patch is a drug-in-adhesive TDS device and, therefore, the drug for delivery is included in the adhesive layer. In other embodiments, the TDS patch is a reservoir-type TDS device and the drug can be included in the adhesive layer or not included in the adhesive layer. Preferably, the elastomer used in the adhesive layer is silicone, a natural rubber, a synthetic rubber (e.g., styrene rubber or polyisobutylene (PIB)), a polyacrylate, polyvinylacetate, polydimethylsiloxane, and combinations thereof. In some embodiments, the adhesive layer has a thickness of about 0.5 mil to about 5 mil. In a preferred embodiment, the adhesive layer has a thickness of about 1 mil to about 3 mil.

In a certain aspect, the adhesive layer of the TDS patch contains PIB. PIB is usually a blend of a high molecular weight PIB (about 450,000 to about 2,100,000 viscosity average molecular weight) and a low molecular weight PIB (about 1,000 to about 450,000 viscosity average molecular weight). Such PIBs are available commercially such as OPPANOL B 100 (viscosity average molecular weight of about 1,170,000) and OPPANOL B 10 (viscosity average molecular weight of about 40,000). In some embodiments, the PIB blend has a ratio of high molecular weight PIB to low molecular weight PIB of about 1:1 to about 1:4. In preferred embodiments, the PIB blend has a ratio of about 1:2 to about 1:3 high molecular weight PIB to low molecular weight PIB. In some embodiments, the PIB blend is mixed with mineral oil, and the mixture contains from about 25% to about 65% PIB. In other embodiments, the PIB is used with a solvent (which is a non-solvent for the drug), including, but not limited to, heptane, hexane, cyclohexane, and the like. In preferred aspects, the PIB is dissolved in heptane. In some embodiments, the solvent contains from about 25% to about 65% PIB; particularly, from about 30% to about 50% PIB.

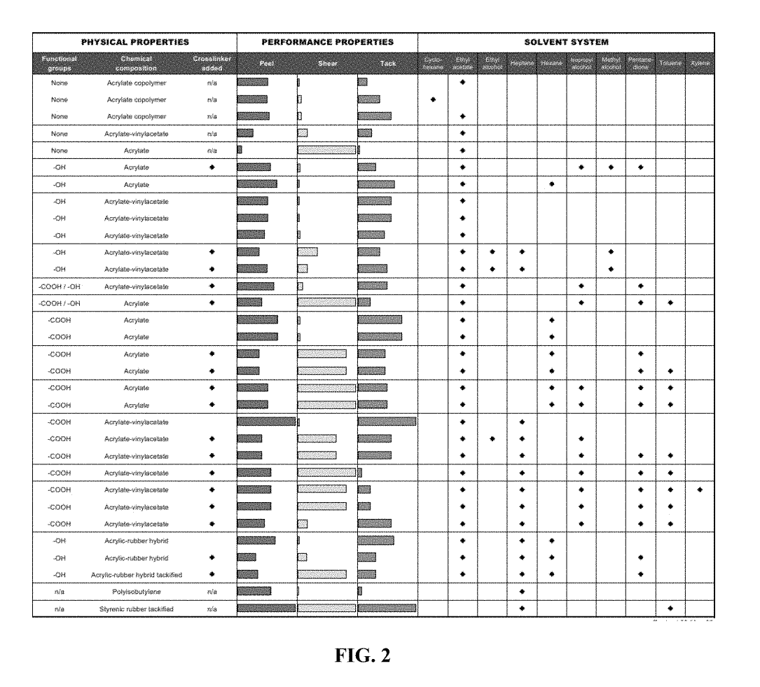

In another aspect, an elastomer of the adhesive layer is a polyacrylate. Polyacrylate PSAs may be prepared in solution by free radical polymerization. In some aspects, the polyacrylate polymer is crosslinked to increase the cohesion of the polymer using art standard methods, including but not limited to thermal curing, UV radiation, or by chemical agents. Suitable crosslinking agents include aluminum acetylacetonate, polybutyl titanate, divinylbenzene, methylene bis-acrylamide, ethylene glycol di(meth)acrylate, ethylene glycol tetra(meth)acrylate, propylene glycol di(meth)acrylate, butylene glycol di(meth)acrylate, trimethylolpropate tri(meth)acrylate, polyisocyanate/polypropylene carbonate mixture, and aliphatic polyisocyanate. Furthermore, the physical properties or performance characteristics (e.g., peel adhesion, tack, and dynamic shear) of the polyacrylate PSA are influenced by the functional groups, such as hydroxyl (—OH) or carboxyl (—COOH) functional groups, and/or the presence of one or more vinyl compounds combined with the polyacrylate PSA. Suitable vinyl compounds include, but are not limited to vinyl acetate, ethyl vinyl ether, vinyl chloride, vinylidene chloride, and acrylonitrile. In some embodiments, the polyacrylate polymers present in the PSA is between about 30% and about 60% by weight; particularly between about 40% about 50% by weight. FIG. 2 provides a non-limiting list of polyacrylate PSAs and the corresponding performance characteristics.

Other PSAs suitable for use in the adhesive layer of the TDS patches used herein include silicone polymers, natural rubbers, styrene rubbers, and the like.

In some embodiments, a TDS patch includes a release liner that protects the adhesive layer and is removed prior to use. In various embodiments, the TDS patch includes a release liner disposed over the adhesive layer and protects the adhesive layer until the time of use and is peeled off before contacting with the intact skin of the individual. Typical release liners are from about 0.5 mils to about 8 mils thick, particularly from about 2 mil to about 4 mils thick, and can be made from thermoplastic materials including transparent fluoropolymer coated polyester, low density polyethylene (LDPE), polyethylene terephthalate (PET), LDPE/PET combinations, and the like. In one embodiment, the release liner is a 2 mil LDPE top release liner (Berry Plastics, Greenville, S.C., USA). In a particular embodiment, the release liner is a 2 mil PET release liner (W-4002, Adhesives Research, Glen Rock, Pa., USA). Other suitable release liners are commercially available and include, e.g., 3 mil SCOTCHPAK 1022 fluoropolymer coated polyester release liner (3M Drug Delivery System, St. Paul, Minn., USA).

The TDS patches provided herein may be configured for therapeutically effective transdermal delivery of a variety of drugs or pharmaceutical agents, including, but not limited to clonidine, scopolamine, oxybutynin, lesopritron, estradiol, levonorgestrel, fentanyl, albuterol, labetalol, atropine, haloperidol, isosorbide dinitrate, nitroglycerin, norethindrone acetate, nicotine, benztropine, secoverine, dexsecoverin, arecoline, buprenorphine, donepezil hydrochloride, donepezil base, lidocaine, selegiline, rivastigmine, methylphenidate, diclofenac, ondansetron, varencicline, oxymorphone, rotigotine, and granisetron.

### Overlay

Also provided herein are overlay patches or overlay films that are placed over the TDS patch to aid in the adhesion of the TDS patch to the intact skin of an individual. In certain aspects, a TDS patch is applied to the intact skin of an individual and begins to loosen over time thereby decreasing the surface area of the TDS patch that contacts the skin and potentially interfering with the release rate of the drug contained in the TDS patch. In some embodiments, a separate overlay that does not contain any drug or pharmaceutical agent, such as a drug-free foam-based overlay, is placed over the patch to improve the adhesion of the TDS patch. In a preferred aspect, the TDS patch is fabricated to include an integrated (or built-in) overlay that is attached to the TDS patch. In these aspects, the integrated overlay will include a backing layer and an adhesive layer, and the surface area of the overlay will be larger than the surface area of the TDS patch to create a drug-free, adhesive-only border or perimeter around the adhesive layer of the TDS patch thereby increasing the total adhesive surface area. In an embodiment, the overlay provides an adhesive-only boarder around the adhesive layer of the TDS patch that is about 1 mm to about 15 mm, e.g., about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm.

FIG. 1C depicts a non-limiting embodiment of an overlay suitable for use herein. As shown in FIG. 1C, the overlay 210 includes an overlay backing layer 220 and an overlay adhesive layer 230. In preferred embodiments, the overlay 210 includes an overlay release liner 240 disposed on the overlay adhesive layer 220 that provides protection for the overlay and is removed prior to integration of the overlay into a TDS patch.

Preferably, the overlay backing layer is an occlusive layer and substantially impermeable to the drug contained in the TDS device. Backing layers or films for use in the overlay of the present disclosure are usually derived from synthetic polymers like polyolefin oils, polyester, polyethylene, polyvinylidine chloride, and polyurethane and may be multilaminates comprising more than one layer of synthetic polymer. Typically, the thickness of the overlay backing layer is from about 0.5 mil to about 5 mils; more particularly, about 1 mil to about 3 mils. In one embodiment, the backing layer is a 2.8 mil multilayer laminate of pigmented polyethylene, aluminum vapor coated polyester, and an ethylene vinyl acetate heat-seal layer (e.g., 3M SCOTCHPAK 9730, 3M Drug Delivery Systems, St. Paul, Minn., USA) or a 2 mil polyester and ethyl vinyl acetate (EVA) polymer (e.g., 3M SCOTCHPAK 9733, 3M Drug Delivery Systems, St. Paul, Minn., USA). In preferred embodiments, the overlay backing layer and the TDS patch backing layer are made from the same materials to avoid affecting the chemistry and release rate of the drug.

In preferred aspects, the overlay will include an adhesive layer, which comprises a PSA containing an elastomer. Preferably, the elastomer used in the overlay adhesive layer is silicone, a natural rubber, a synthetic rubber (e.g., styrene rubber or PIB), a polyacrylate, polyvinylacetate, polydimethylsiloxane, and combinations thereof, each of which are described in more detail elsewhere herein. The composition of the adhesive layers in both the TDS patch and the overlay potentially affects the chemistry of the device and therefore the release rate of the drug. Thus, in more preferred embodiments, the adhesive layer of the overlay will include the same or substantially the same elastomer as in the adhesive layer of the TDS patch. In some embodiments, the overlay adhesive layer has a thickness of about 0.5 mil to about 5 mil. In a preferred embodiment, the overlay adhesive layer has a thickness of about 1 mil to about 3 mil.

It is another aspect of the invention to fabricate an overlay for integration in a TDS patch that has the same or substantially the same PSA composition as in the TDS patch. In a more preferred aspect, the peel adhesion of the overlay is increased as compared to the TDS patch. In some embodiments, the increased peel adhesion of the overlay adhesive layer is accomplished by altering the chemical composition of the elastomers. For instance, as shown in FIG. 2, the peel adhesion of polyacrylates can be increased by including polyacrylates with carboxyl functional groups in place of polyacrylates with hydroxyl functional groups. In other embodiments, the overlay adhesive layer includes PIB. In such embodiments, the peel adhesion is increased by adjusting the ratio of high molecular weight PIB to low molecular weight PIB in the PIB blend (low molecular weight PIBs tend to have tackifying abilities, whereas high molecular weight PIBs increase cohesion). The performance characteristics of different PIBS and PIB blends suitable for use in adhesives is described further in Willenbacher & Lebedeva, “Polyisobutene-Based Pressure-Sensitive Adhesives”, in TECHNOLOGY OF PRESSURE-SENSITIVE ADHESIVES AND PRODUCTS 4.1-4.18 (2008), the entire content of which is incorporated herein by reference. In other embodiments, the % wt of the PSA in the solvent is increased as compared to the patch adhesive layer. In yet other embodiments, the peel adhesion of the overlay adhesive layer includes the addition of tackifying resins. Suitable tackifying resins include terpene resins, hydrocarbon resins (e.g., aliphatic hydrocarbon resins, aromatic hydrocarbon resins, dicyclopentadiene resins, and mixtures thereof), rosin resins, and terpene-phenol resins.

It is generally known in the art that there is an inverse relationship between peel adhesion and dynamic shear of a PSA. In other words, if one increases the peel adhesion of the PSA, the shear decreases, and vice versa. Thus, while increasing the peel adhesion of a TDS patch will increase its ability to stick to a substrate, such as the intact skin of a patient, the TDS patch will lose the ability to remain adhered to the substrate in response to movement. Surprisingly, however, the TDS patches with integrated overlays provided herein improve the overall peel adhesion of the TDS device without significantly decreasing the tack or dynamic shear (see Examples 4 and 5).

In some embodiments, the overlay of the invention includes a release liner that protects the overlay adhesive layer and is removed prior to disposing onto the TDS patch. Typical overlay release liners are from about 0.5 mils to about 8 mils thick, particularly from about 2 mil to about 4 mils thick, and can be made from thermoplastic materials including transparent fluoropolymer coated polyester, low density polyethylene (LDPE), polyethylene terephthalate (PET), LDPE/PET combinations, and the like. In one embodiment, the overlay release liner is a 2 mil LDPE top release liner (Berry Plastics, Greenville, S.C., USA). In a particular embodiment, the release liner is a 2 mil PET release liner (W-4002, Adhesives Research, Glen Rock, Pa., USA). Other suitable release liners are commercially available and include, e.g., 3 mil SCOTCHPAK 1022 fluoropolymer coated polyester release liner (3M Drug Delivery Systems, St. Paul, Minn., USA).

Fabrication of the Transdermal Drug Delivery Device with Integrated Overlay

In another aspect, this invention features methods for manufacturing overlays for integration with TDS patches and methods for manufacturing TDS patches with integrated overlays. The overlays provided herein have at least a backing layer or film and an adhesive layer (see, e.g., FIG. 1C). The overlays preferably are designed to be adhered to the backing layer of the TDS patch and are thus “built-in”. While any PSA may be used to construct the adhesive layer of the overlay, it is preferred that the PSA used in the overlay adhesive layer is the same or substantially the same as the PSA in the TDS patch adhesive layer.

The TDS patches for use with the integrated overlays provided herein are produced by art-standard methods. For instance, a reservoir-type TDS patch may be fabricated by first suspending the pharmaceutical agent in an adhesive to produce a drug reservoir. The adhesive for the adhesive layer may be coated onto a substrate, such as a polyester release liner, by solution casting, reverse roll coating, doctor knife coating (or knife-over-roll coating), extrusion, and the like, which is then cured to remove any solvents used to dissolve or suspend the PSA mixture. Curing can be performed using any suitable method known in the art, such as thermal or heat curing, chemical curing, evaporation, or UV exposure. If a rate-controlling membrane is used (e.g., microporous membrane comprising polypropylene), the it may be laminated (e.g., pressure laminated) to the exposed adhesive. The drug reservoir is then sandwiched between a standard backing layer and the rate-controlling membrane and adhesive layer assembly, which can be laminated or heat-sealed or other using art standard methods. The fabrication process is typically carried out on a rotary press wherein the TDS patches may be cut to size using, e.g., standard die cutting equipment, or rolled into a core and stored. Various methods for fabricating both reservoir-type TDS patches and drug-in-adhesive TDS patches are described in, e.g., U.S. Pat. No. 5,965,154, U.S. Pat. No. 9,248,104, WO 00/24386, and US 2014/0005617 A1.

In a particular aspect, methods for fabricating an overlay suitable for integrating with a TDS patch are provided. Overlays can be fabricated using any standard equipment typically used in the art, including, but not limited to extruding, doctor knife coating (or knife-over-roll coating), reverse roll coating, size pressing, coil coating, and foil coating (e.g., doctor knife coating system, Werner Mathis USA Inc., Concord, N.C., USA). In an embodiment, the adhesive layer composition is prepared with a suitable PSA elastomer as described in more detail elsewhere herein. In a preferred embodiment, the elastomer used in the overlay adhesive layer is the same or substantially the same as the elastomer contained in the adhesive layer of the TDS patch to which the overlay will be integrated. In a more preferred embodiment, the overlay adhesive layer will have increased peel adhesion as compared to the TDS patch adhesive layer. The adhesive mixture is then coated onto a suitable substrate, such as a backing layer or a release liner. In a preferred embodiment, the adhesive mixture is coated onto a release liner at a targeted thickness of about 0.5 mil to about 5 mil, e.g., about 0.5 mil, 0.6 mil, 0.7 mil, 0.8 mil, 0.9 mil, 1.0 mil, 1.1 mil, 1.2 mil, 1.3 mil, 1.4 mil, 1.5 mil, 1.6 mil, 1.7 mil, 1.8 mil, 1.9 mil, 2.0 mil, 2.1 mil, 2.2 mil, 2.3 mil, 2.4 mil, 2.5 mil, 2.6 mil, 2.7 mil, 2.8 mil, 2.9 mil, 3.0 mil, 3.1 mil, 3.2 mil, 3.3 mil, 3.4 mil, 3.5 mil, 3.6 mil, 3.7 mil, 3.8 mil, 3.9 mil, 4.0 mil, 4.1 mil, 4.2 mil, 4.3 mil, 4.4 mil, 4.5 mil, 4.5 mil, 4.7 mil, 4.8 mil, 4.9 mil, and 5.0 mil. In a preferred embodiment, the targeted thickness is between about 1.0 mil and 3.0 mil. The adhesive layer is then cured to remove the solvents. In a preferred embodiment, the adhesive layer and release liner assembly is baked at a temperature range of about 60° C. to about 160° C.; particularly, the temperature range is from about 80° C. to about 150° C. In some embodiments, the curing is performed as a series of increasing temperature ranges. By way of example only and not intending to be limiting, in one embodiment, the curing is performed as a series of three temperature zones, e.g., a first temperature zone having a temperature range of about 80° C. to about 100° C.; a second temperature zone having a temperature range of about 100° C. to about 120° C., and a third temperature zone having a temperature range of about 120° C. to about 150° C. In yet other embodiments, the curing process is performed in a series of four temperature zones or more. The top membrane, e.g., backing layer, is then laminated onto the adhesive layer, which can then be rolled onto a core for storage or loaded onto a rotary press and die cutter for fabrication of the TDS patch with integrated overlay. Alternatively, the adhesive layer may be extruded or coated onto a backing layer and immediately integrated into the TDS patch. Suitable backing layer and release liner materials are described in more detail elsewhere herein.

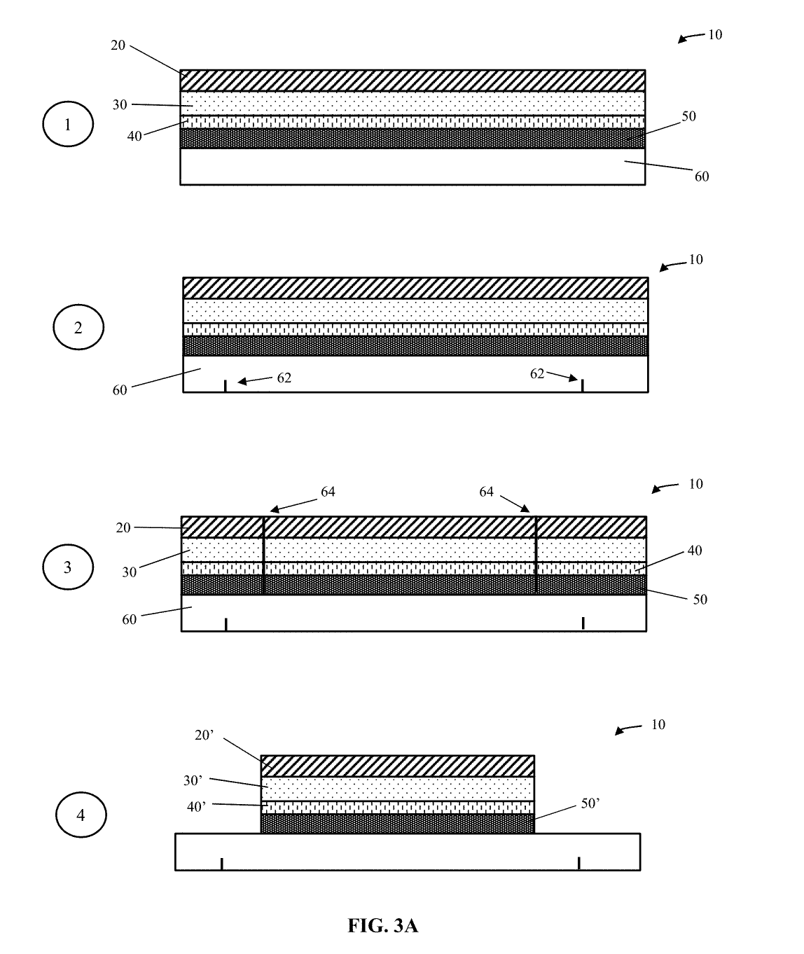

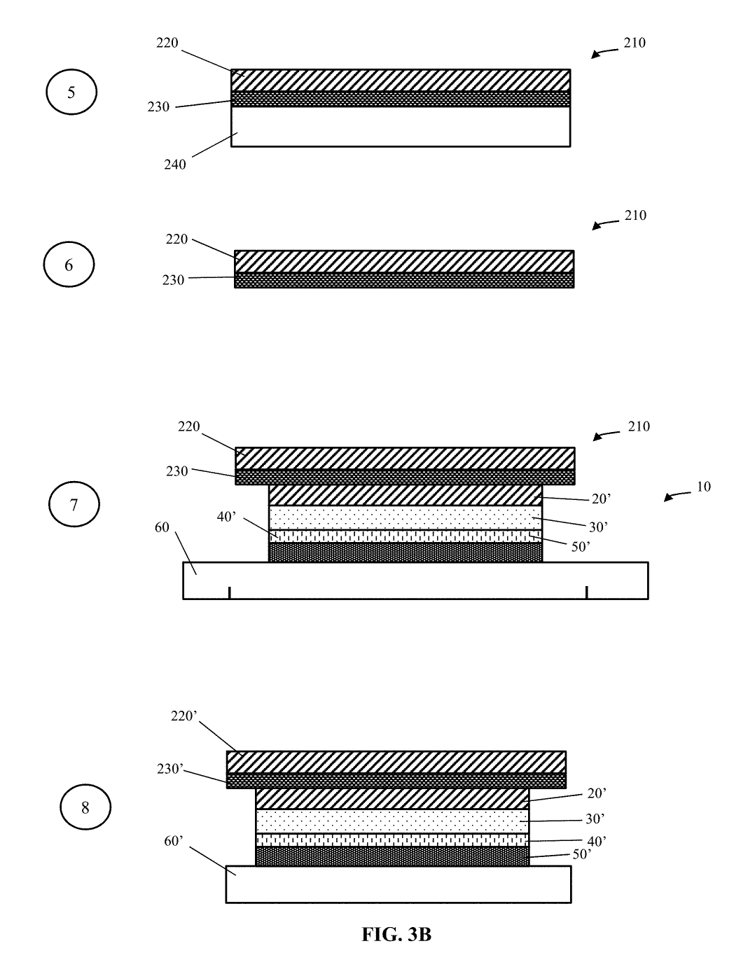

To convert a TDS patch to a TDS patch with integrated overlay, it is preferable to use a drug roll for ease of manufacturing. The drug roll may contain the assembled layers for cutting any type of TDS patch, including, but not limited to, a reservoir-type TDS patch (e.g., a release liner, an adhesive layer, a rate-controlling membrane, a drug reservoir, and a backing layer) or a drug-in-adhesive TDS patch (e.g., a release liner, an adhesive-drug layer, and a backing layer). The conversion of a TDS patch to one with an integrated overlay can be performed on an art standard rotary press or other similar device as a single pass process or, alternatively, the various stages of the lamination and cutting of the TDS patches and overlays can be done on one or more passes using intermediate builds. Preferably, the fabrication process begins with a wound roll of TDS patch layers in a large sheet that has not yet been cut into the final patch product sizes. FIGS. 3A and 3B provide a diagram of an embodiment of a single pass fabrication process integrating an overlay on a reservoir-type TDS patch, although it will be readily appreciated by the skilled artisan that the fabrication method may comprise a drug-in-adhesive TDS patch or any other type of TDS patch. As shown in FIG. 3A, the various layers of the TDS patch sheet 10 include the backing layer 20, the drug reservoir 30, the rate-controlling membrane 40, the adhesive layer 50, and a release liner 60. The drug roll is loaded onto a pay-off shaft and threaded through all the press stations. In addition to the drug roll, FIG. 3B (#5) shows an overlay 210 having a backing layer 220, an adhesive layer 230, and a release liner 240, which is also loaded a payoff shaft on the press.

The first station includes a back-score die that back-scores 62 only the release liner 60 (see FIG. 3A #2). The TDS patch layers then pass to the second cutting station (#3) where a kiss-cut die cuts 64 through each of the backing layer 20, drug reservoir 30, rate-controlling membrane 40, and adhesive layer 50 down to the release liner 60, but leaves the release liner 60 intact. As the TDS patch layers move out from station two, the excess drug liner is peeled back to the face of the kiss-cut and away from the TDS patch layers that will be used in the final patch (i.e., the backing layer 20′, the drug reservoir 30′, the rate-controlling membrane 40′, and the adhesive layer 50′).

While the TDS patch layers are moving through the press, the overlay 210 is being processed for being laminated onto the TDS patch. First, the release liner 240 is peeled away from the overlay to expose the adhesive layer 230 (see FIG. 3B #6). At the third press station, the overlay layers are threaded under a peel bar and over a laminating roll that laminates the overlay onto the TDS patch. Shown in FIG. 3B step 7 is the overlay adhesive layer 230 contacting the backing layer 20 of the TDS patch. The laminated materials are then passed to the fourth press station where a steel to steel die cuts through the overlay backing layer 220′, the overlay adhesive layer 230′, and the patch release liner 60′ to create the final size and shape of the TDS patch with integrated overlay (see FIG. 3B #8).

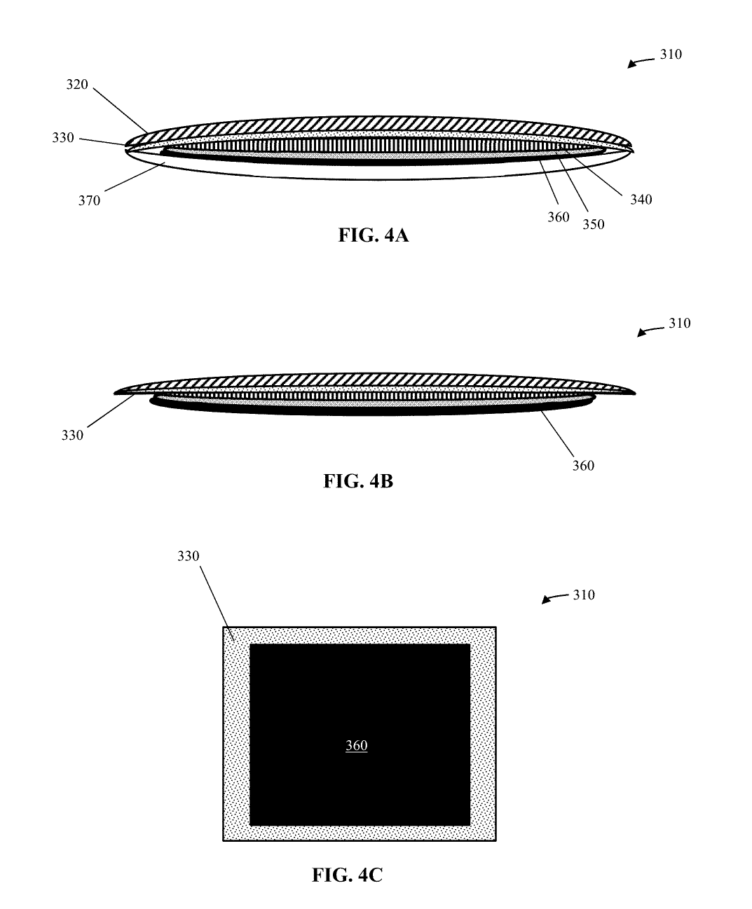

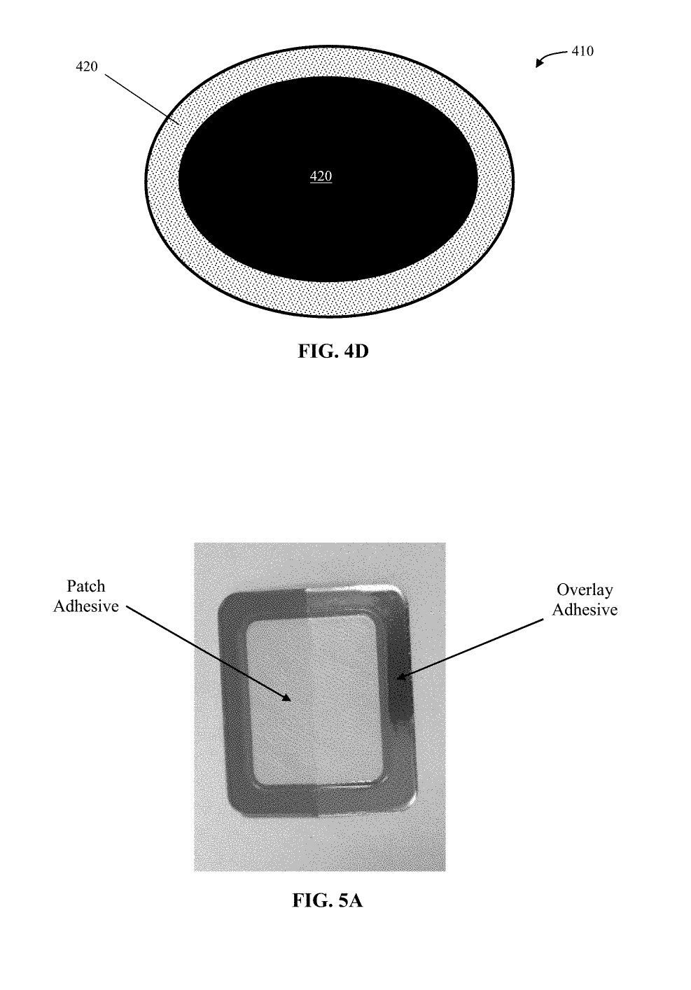

FIG. 4A depicts a cross sectional diagram of a non-limiting, exemplary embodiment TDS patch with integrated overlay. The TDS patch with integrated overlay 310 includes an overlay comprising an overlay backing layer 320 and an overlay adhesive layer 330 that has been laminated to the TDS patch, which includes a patch backing layer 340, an drug reservoir and rate-controlling membrane 350, patch adhesive layer 360, and a patch release liner 370. When ready for use, the patch release liner 370 is removed thereby exposing the patch adhesive layer 360 for applying to the intact skin. As can be seen in FIGS. 4B and 4C, the overlay has an adhesive layer 330 (and backing layer 320) that is wider and longer than the TDS patch and creates a drug-free adhesive border that surrounds the TDS patch adhesive layer 360. FIG. 4D depicts another embodiment of the TDS patch with integrated overlay 410. Shown in FIG. 4D is the adhesive layer 410 of the overlay as compared to the adhesive layer 420 of the TDS patch. In an embodiment, the overlay provides an drug-free, adhesive-only boarder around the adhesive layer of the TDS patch that is about 1 mm to about 15 mm, e.g., about 1 mm, 2 mm, 3 mm, 4 mm, 5 mm, 6 mm, 7 mm, 8 mm, 9 mm, 10 mm, 11 mm, 12 mm, 13 mm, 14 mm, 15 mm. Preferably, the drug-free, adhesive-only border is about 5 mm to about 9 mm. In an embodiment, the TDS patch with integrated overlay is applied to the skin for at least about 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, 48 hours, 72 hours, 4 days, 5 days, 6 days, 7 days, 8 days, or more, without lifting, loosing, or peeling.

The TDS patches with integrated overlay provided herein may be configured for therapeutically effective transdermal delivery of a variety of drugs or pharmaceutical agents, including, but not limited to clonidine, scopolamine, oxybutynin, lesopritron, estradiol, levonorgestrel, fentanyl, albuterol, labetalol, atropine, haloperidol, isosorbide dinitrate, nitroglycerin, norethindrone acetate, nicotine, benztropine, secoverine, dexsecoverin, arecoline, buprenorphine, donepezil hydrochloride, donepezil base, lidocaine, selegiline, rivastigmine, methylphenidate, diclofenac, ondansetron, varencicline, oxymorphone, rotigotine, and granisetron.

The TDS patches with integrated overlay described herein and fabricated by the methods provided have improved peel adhesion and a larger overall adhesive surface area. Preferably, the overlay and the TDS patch have the same or substantially the same adhesive layer compositions and backing layer compositions. In a more preferred embodiment, the overlay will have improved peel adhesion due to modifications in the PSA composition while using the same or substantially the same elastomers as the TDS patch. Furthermore, the integral overlay is firmly anchored to the backing layer such that it will not interfere with the TDS patch adhesive layer or drug layer and will therefore have minimal impact on the pharmacokinetics of the drug release rate. Further, by providing a drug-free, adhesive perimeter, the peel adhesion and shear of the overlay can be increased without influencing the drug release rate.

### Kits and Packaging

The TDS devices with integrated overlay can be packaged for storage, distribution and use in accordance with any suitable protocol well known to the skilled artisan. For instance, the devices can be packaged into individual or multi-compartment packs or envelopes for storage and delivery.

Thus, another aspect of the invention comprises kits for use in practice of the present invention. The kits comprise one or more TDS devices with integrated overlay and instructions for use of the devices to transdermally deliver a therapeutically effective amount of the drug in accordance with the methods described herein.

The following examples describe the invention in greater detail. They are intended to illustrate, rather than to limit, the invention.

### Example 1. Fabrication of Overlays

An overlay for integration was fabricated for a fentanyl TDS patch, which was a drug-in-adhesive type patch (see FIG. 1B). The backing layer of the fentanyl TDS patch was a 2 mil polyester and ethyl vinyl acetate (EVA) polymer (i.e., 3M SCOTCHPAK 9733 polyester backing film laminate, 3M Drug Delivery Systems, St. Paul, Minn., USA). The drug/adhesive layer included a fentanyl base, copovidone, and a polyacrylate adhesive. The fentanyl TDS patch included a 2 mil release liner made of PET film (i.e., W-4002, Adhesives Research, Glen Rock, Pa., USA).

The overlay was fabricated to include the same backing layer as the fentanyl TDS patch and an adhesive made from a polyacrylate dissolved in ethyl acetate. To increase the peel adhesion, a polyacrylate composition with hydroxyl functional groups was chosen. However, alternatively the peel adhesion of the polyacrylate PSA can be altered, e.g., by the use of polyacrylates with other functional groups or by the addition of a crosslinker or EVA (see FIG. 2). The adhesive for the overlay did not contain copovidone or fentanyl.

The adhesive was coated onto a 2 mil PET release liner (W-4002, Adhesives Research, Glen Rock, Pa., USA) at a targeted thickness of about 2.2 mil using a doctor knife coating (or knife-over-roll coating) system (Werner Mathis USA Inc., Concord, N.C., USA). The assembly was then heat cured using art standard techniques to remove the solvent. A 3M SCOTCHPAK 9733 backing layer was laminated onto the adhesive layer to produce an overlay for a fentanyl TDS patch with integrated overlay.

Another overlay for integration was fabricated for a clonidine TDS patch, for example, as described in U.S. Pat. No. 4,201,211, the entire contents of which is hereby incorporated herein by reference. The clonidine TDS patch was a drug reservoir type patch (see FIG. 1A). The backing layer was a 2.8 mil multilayer laminate of included pigmented polyethylene, aluminum vapor coated polyester, and an ethylene vinyl acetate heat-seal layer (3M SCOTCHPAK 9730, 3M Drug Delivery Systems, St. Paul, Minn., USA). The drug reservoir contained a mixture of clonidine, mineral oil, colloidal silicon dioxide, and polyisobutylene (PIB). The clonidine TDS patch also included a microporous polypropylene membrane. The adhesive layer contained an amount of clonidine (less than that of the drug reservoir) mixed with mineral oil and PIB. Finally, the clonidine TDS patch included a 2 mil release liner made from PET film (i.e., W-4002, Adhesives Research, Glen Rock, Pa., USA).

The overlay was fabricated to include the same backing layer as the clonidine TDS patch and an adhesive made from the same type of elastomer as the clonidine TDS patch (i.e., PIB dissolved in heptane). To increase peel adhesion, a tackifying resin] was added to the PIB mixture. Alternatively, the ratio of high viscosity average molecular weight polymers to low viscosity average molecular weight polymers could have been altered to enhance the peel adhesion of the PIB mixture instead of, or in addition to, the tackifying resin. The adhesive for the overlay did not contain mineral oil or clonidine.

The adhesive was coated onto a 2 mil PET release liner (W-4002, Adhesives Research, Glen Rock, Pa., USA) at a targeted thickness of about 1.0 mil, 1.8 mil, or 2.0 mil using a doctor knife coating system (Werner Mathis USA Inc., Concord, N.C., USA). The assembly was then heat cured using art standard techniques to remove the solvent. A 3M SCOTCHPAK 9730 backing layer was laminated onto the adhesive layer to produce an overlay for an clonidine TDS patch with integrated overlay.

### Example 2. Fabrication of a Clonidine TDS Patch with Integrated PIB Overlay

In a non-limiting exemplary process, the conversion of a TDS patch to a TDS patch with an integrated overlay was done using a rotary press. This particular embodiment was a single pass process that enabled the TDS patch conversion without requiring the building of intermediate rolls. The rotary press included four processing stations and several die cutters. First, a roll of the clonidine drug and other desired laminates were assembled and loaded onto the rotary press. The clonidine drug assembly roll contained a clonidine TDS patch assembly as described in Example 1. The clonidine drug assembly roll was loaded onto a pay-off shaft and threaded through all the press stations and attached to an empty core on a rewind shaft. At the first station, a back-score die scored only the release liner of the drug assembly to aid in its removal by the end-user. The scored drug assembly was then moved to the second station, where a kiss-cut (cut-to-liner) die cut through the backing layer, drug reservoir, control membrane, and adhesive layer to the correct size for the final product, but left the release liner intact. Upon exiting station two, the excess material resulting from the kiss-cut (the backing layer, drug reservoir, control membrane, and adhesive layer that will not be used in the final product) was cut free from the release liner and peeled back to the face of the kiss-cut where it was threaded over the waste guide rollers. From there, the waste matrix of drug laden adhesive accumulated on a rewind shaft. The back-scored and cut drug assembly was then moved to the third station for assembly with the overlay.

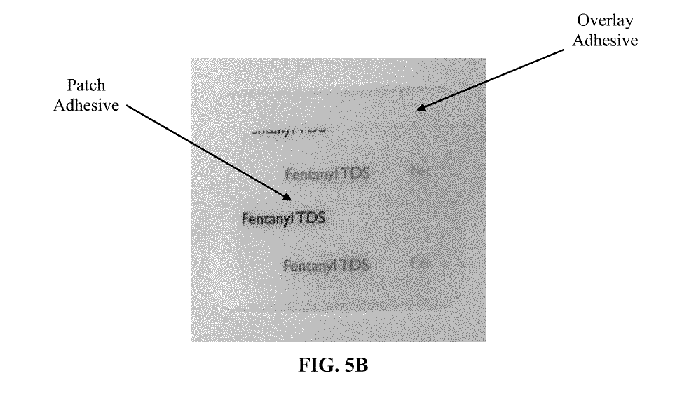

The PIB overlay material that was fabricated in Example 1 was placed on a payoff shaft above station three. The PIB overlay was separated from its release liner and threaded under the peel bar, over the laminating roll, and into contact with the drug patch release liner such that the PIB overlay material completely covered the backing layer (i.e., the clonidine TDS patch). The PIB overlay material was then laminated onto the exposed portions of the patch release liner. The PIB overlay release liner was threaded through waste guide rollers on to a rewind shaft used to accumulate the release liner for disposal. The laminated assembly was then carried to station four, where a steel to steel (through cut) die cut through the PIB overlay and clonidine TDS release liner creating an occlusive finished patch with a border adhesive. The dimension of the steel to steel cut was larger than the dimensions of the kiss-cut in order to provide a border adhesive of about 7 mm. Thus, the finished clonidine TDS patch had an occlusive border adhesive overlay incorporated into the overall patch with an easily removable release liner. FIG. 5A shows a photograph of an exemplary clonidine TDS patch with integrated overlay.

### Example 3. Testing Methods for Determining the Adhesive Physical Properties

Peel Adhesion Test.

Peel adhesion is a common test in the pharmaceutical and textile industries to determine the adhesive strength of a pressure-sensitive materials or the strength of the adhesive bond between two materials. The adhesive strength is a measure of the materials' resistance to separation from one another after the adhesive has been applied. A 180° peel test requires the adhesive to be placed against a steel plate with the bonded area between the tape and plate placed vertically between the test grips.

Peel adhesion testing was performed on TDS patches, overlays, or TDS patches with integrated overlays using the 180° Peel Adhesion Test according to the American Society for Testing and Materials (ASTM) International standards D3330, Standard Test Method for Peel Adhesion of Pressure-Sensitive Tape (2010); D5375, Standard Test Methods for Liner Removal at High Speeds from Pressure-Sensitive Label Stock (2011); and D6252, Standard Test Method for Peel Adhesion of Pressure-Sensitive Label Stocks at a 90° Angle (2011), the entire contents of each are incorporated herein by reference.

Briefly, an overlay (cut to the size of about 1 inch wide by about 3.5 inches long) or intact TDS patch was tested. Masking tape was cut to about 1 inch wide by about 6 inches long. The release liner on the overlay (if present) or TDS patch was slowly removed by about 0.5 inches from one end, and the masking tape was attached lengthwise to the exposed adhesive. The remaining release liner was slowly removed from the test sample, and the sample was then attached to a clean steel plate such that the sample was about 1 inch from the edge of the plate. A rubber padded roller was rolled over the sample. The plate was attached to the lower jaw accessory of a TA-XT Plus Texture Analyzer (Stable Micro Systems Ltd., Godalming, Surrey, UK) and the masking tape was attached to the upper jaw accessory. The “180° peel adhesion test” was then run and the average peel adhesion for each sample was reported in grams per inch (g/in.).

Probe Tack Test.

Probe tack is a common test in the pharmaceutical and textile industries to determine the initial adhesion and release of pressure-sensitive adhesive materials using a probe. Probe tack testing was performed on TDS patches, overlays, or TDS patches with integrated overlays using the probe tack test according to the ASTM International standard D2979, Standard Test Method for Pressure-Sensitive Tack of Adhesives Using an Inverted Probe Machine (2009), the entire content of which is incorporated herein by reference.

Briefly, the overlay or TDS patch was cut to a size of about 1 inch wide by about 1.5 inches long. A tack plate from a TA-XT Plus Texture Analyzer with a tack probe (Stable Micro Systems Ltd., Godalming, Surrey, UK) was placed on a flat surface with the grooved side facing front. The overlay or TDS patch was applied the plate by slowly removing the release liner and placing onto the tack plate such that the overlay or TDS patch completely covered two adjacent holes. A rubber padded roller was rolled over the sample, and the tack plate was slid onto the platform of the TA-XT Plus Texture Analyzer so that the grooved side was facing front and the samples were on the down side of the plate exposing the holes to the tack probe. The “tack test” was then run and the average tack for each sample was reported in grams (g).

Dynamic shear Test. To determine the ability of an adhesive to stick to a substrate when subject to dynamic forces or movement, a dynamic shear test was performed on TDS patches, overlays, or TDS patches with integrated overlays according to the ASTM International standards D6463, Standard Test Method for Time to Failure of Pressure Sensitive Articles Under Sustained Shear Loading (2012); and D3654, Standard Test Methods for Shear Adhesion of Pressure-Sensitive Tapes (2011), the entire contents of each are incorporated herein by reference.

Briefly, two overlays or two TDS patches were each cut to a size of about 1 cm in width (the length of the overlays and patches were greater than 1 cm). The release liners were slowly removed and each pair of overlays or each pair of transdermal patches was attached with the adhesive sides making contact. Masking tape was attached to the opposite ends at the longer sides of the assembly. The assembly was then cut to about 2 cm×1 cm on each side of the 1 cm×1 cm sample. A rubber padded roller was rolled over the assembly, and one end of the assembly was attached to the lower jaw accessory of a TA-XT Plus Texture Analyzer (Stable Micro Systems Ltd., Godalming, Surrey, UK) with the other end of the assembly attached to the upper jaw accessory. The “dynamic shear test” was then run for at least 1,000 seconds. The average maximum force for each sample was reported in grams (g).

### Example 4. Physical Data for Clonidine TDS Patches with Integrated Overlays

Adhesion performance of the CATAPRES-TTS-3, an RLD brand, 7-day, 0.3 mg/day clonidine TDS patch (Boehringer Ingelheim Pharmaceuticals, Ridgefield, Conn., USA), was compared to a generic 7-day, 0.3 mg/day clonidine TDS patch (Core Tech Solutions, Inc., East Windsor, N.J., USA) and to the generic clonidine TDS patch with an integrated PIB overlay fabricated according to the methods described herein (e.g., Examples 1 and 2 and FIG. 5A). Each of the CATAPRES-TTS-3 TDS patch, generic clonidine TDS patch, and PIB overlay included a PIB adhesive and a backing layer made from a multilaminate pigmented polyethylene, aluminum vapor coated polyester, and an EVA heat-sealed layer, such as the commercially available 2.8 mil 3M SCOTCHPAK 9730 backing polyester film laminate (3M Drug Delivery Systems, St. Paul, Minn., USA). In addition, the CATAPRES TT-3 TDS patch is sold with a separate foam overlay to place over the patch in the event that it begins to lift or loosen during wear. Thus, adhesion measurements of the foam overlay with the clonidine TDS patch and the CATAPRES TT-3 TDS patch were obtained and compared to the clonidine TDS patch with integrated PIB overlay. Specifically, 180° peel adhesion, probe tack, and dynamic shear was measured for each according to the procedures described in Example 3. As summarized in Table 1, the clonidine TDS patch with integrated PIB overlay exhibited superior peel adhesion values as compared to both either TDS patch alone or with the foam overlay.

TABLE 1
Foam Overlay Compared to PIB Overlay

| | CATAPRES TT-3 | | | Clonidine TDS | | | |
|---|---|---|---|---|---|---|---|
| | | Foam | Patch with | | Patch with | PIB | Patch with |
| | | overlay | foam | | foam | overlay | integrated |
| | Patch only | only | overlay | Patch only | overlay | only | PIB overlay |
| Peel | 105.7 | 357.3 | 331.0 | 179.0 | 456.7 | 2468.5 | 1508.8 |
| Adhesion | | | | | | | |
| (g) | | | | | | | |
| Peel | 92.3 | 179.9 | 166.6 | 156.4 | 230.2 | 2470.7 | 892.0 |
| Adhesion | | | | | | | |
| (g/inch) | | | | | | | |
| Probe | 146.2 | 105.3 | N/A | 139.5 | N/A | 195.4 | N/A |
| Tack (g) | | | | | | | |
| Shear (g | 97.3 | 394.1 | N/A | 95.0 | N/A | 218.9 | N/A |
| max | | | | | | | |
| force) | | | | | | | |

N/A, not applicable.

### Example 5. Physical Data for Fentanyl TDS Patch with Integrated Overlay

Adhesion performance of the DURAGESIC, a reference listed drug (RLD) brand, 72-hour, 25 mg/h fentanyl TDS patch (Alza Corporation, Mountain View, Calif., USA), was compared to a generic 72-hour, 25 mg/h fentanyl TDS patch (Core Tech Solutions, Inc., East Windsor, N.J., USA) and to the generic fentanyl TDS patch with an integrated polyacrylate overlay fabricated according to the methods described herein (e.g., Examples 1 and 2 and FIG. 5B). Each of the DURAGESIC TDS patch, generic fentanyl TDS patch, and polyacrylate overlay included a polyacrylate adhesive and a backing layer made from polyester and ethyl vinyl acetate (EVA) polymer, such as the commercially available 2 mil 3M SCOTCHPAK 9733 polyester backing film laminate (3M Drug Delivery Systems, St. Paul, Minn., USA). Specifically, 180° peel adhesion, probe tack, and dynamic shear was measured for each according to the procedures described in Example 3. As summarized in Table 2, the fentanyl TDS patch with integrated polyacrylate overlay exhibited superior peel adhesion values as compared to the fentanyl TDS patch alone and had comparable values to the DURAGESIC TDS patch.

TABLE 2
Performance of the Polyacrylate Overlay

| | | Fentanyl TDS | | |
|---|---|---|---|---|
| | | | | Patch with |
| | DURAGESIC | | | integrated |
| | TDS (patch | | Polyacrylate | polyacrylate |
| | only) | Patch only | overlay only | overlay |
| Peel | 957.4 | 247.5 | 981.6 | 954.7 |
| Adhesion | | | | |
| (g) | | | | |
| Peel | 936.2 | 242.0 | 982.5 | 564.4 |
| Adhesion | | | | |
| (g/in.) | | | | |
| Probe | 215.4 | 192.5 | 293.1 | 346.9 |
| Tack (g) | | | | |
| Shear (g | 739.3 | 1084.7 | 686.8 | 863.6 |
| max | | | | |
| force) | | | | |

The present invention is not to be limited in scope by the specific embodiments disclosed in the examples, which are intended as illustrations of several aspects of the invention. Any embodiments that are functionally equivalent are within the scope of this invention. Indeed, various modifications of the invention in addition to those shown and described herein will become apparent to those skilled in the art and are intended to fall within the scope of the appended claims.

I claim:

1. A method of increasing adhesion of a transdermal drug delivery patch, the method comprising:
(a) providing a transdermal drug delivery patch, the drug delivery patch comprising a patch adhesive layer and a patch backing layer, wherein the patch adhesive layer comprises a first elastomer;
(b) fabricating an overlay comprising the steps of:
(i) coating an overlay adhesive layer on an overlay release liner;
(ii) laminating an overlay backing layer onto the overlay adhesive layer, wherein: (A) the overlay adhesive layer comprises a second elastomer; (B) the second elastomer is substantially the same elastomer as the first elastomer; and (C) the overlay adhesive layer has a surface area that is greater than a surface area of the patch adhesive layer;
(c) removing the overlay release liner whereby the overlay adhesive layer is exposed; and
(d) integrating the transdermal delivery patch and the overlay, the integrating comprising disposing the overlay on the patch backing layer, wherein the overlay adhesive layer contacts the patch backing layer to produce a transdermal drug delivery patch with integrated overlay having an extended adhesive surface area;
wherein the transdermal drug delivery patch with integrated overlay has an increased peel adhesion as compared to the transdermal drug delivery patch.

2. The method of claim 1, wherein the patch backing layer has a first polymer composition, wherein the overlay backing layer has a second polymer composition, wherein the first polymer composition and second polymer composition comprise substantially the same polymers, and wherein both the first polymer composition and the second polymer composition comprise one or more synthetic polymers selected from the group consisting of polyolefin oils, polyester, polyethylene, polyvinylidine, chloride, and polyurethane.

3. The method of claim 1, wherein the overlay is adhered to the transdermal drug delivery patch by heat curing.

4. The method of claim 1, wherein the transdermal drug delivery patch with integrated overlay has a peel adhesion that is at least 1.5-fold greater as compared to the transdermal drug delivery patch and wherein the overlay adhesive layer has a thickness of at least 1 mil.

5. The method of claim 1, wherein the first elastomer, the second elastomer or both the first elastomer and the second elastomer are selected from the group consisting of polyacrylate, polyisobutylene (PIB), natural rubber, silicone, and styrene rubber.

6. The method of claim 5, wherein the first elastomer is a polyacrylate comprising hydroxyl functional groups, and wherein the second elastomer is a polyacrylate comprising hydroxyl functional groups.

7. The method of claim 6, further comprising, prior to coating the overlay adhesive layer onto the overlay backing layer, increasing the peel adhesion of the overlay adhesive layer as compared to the patch adhesive layer.

8. The method of claim 7, wherein increasing the peel adhesion of the overlay adhesive layer comprises adding a tackifying resin to the overlay adhesive layer.

9. The method of claim 1, wherein:
(i) the transdermal delivery patch is a drug-in adhesive transdermal patch, and wherein the patch adhesive layer comprises a therapeutically effective amount of a drug; or
(ii) the transdermal drug delivery patch is a reservoir-type transdermal patch comprising a drug reservoir, and wherein the drug reservoir comprises a therapeutically effective amount of a drug.

10. The method of claim 9, wherein the drug is selected from the group consisting of clonidine, scopolamine, oxybutynin, lesopritron, estradiol, levonorgestrel, fentanyl, albuterol, labetalol, atropine, haloperidol, isosorbide dinitrate, nitroglycerin, norethindrone acetate, nicotine, benztropine, secoverine, dexsecoverin, arecoline, buprenorphine, donepezil hydrochloride, donepezil base, lidocaine, selegiline, rivastigmine, methylphenidate, diclofenac, ondansetron, varencicline, oxymorphone, rotigotine, and granisetron.

11. The method of claim 1, further comprising contacting the patch adhesive layer of the transdermal drug delivery patch with integrated overlay to skin of an individual.

12. A transdermal drug delivery patch with integrated overlay comprising:
(a) a transdermal drug delivery patch comprising a patch adhesive layer and a patch backing layer, wherein the patch adhesive layer comprises a first elastomer and the patch backing layer comprises a first polymer composition; and
(b) an overlay disposed on the patch backing layer, the overlay comprising an overlay backing layer and an overlay adhesive layer, wherein the overlay adhesive layer comprises a second elastomer that is substantially the same elastomer as the first elastomer; wherein the overlay adhesive layer has a surface area that is greater than a surface area of the patch adhesive layer, and wherein the overlay adhesive layer is attached to the patch backing layer;
wherein the transdermal drug delivery patch with integrated overlay has an extended adhesive surface area and an increased peel adhesion as compared to the transdermal drug delivery patch.

13. The transdermal drug delivery patch with integrated overlay of claim 12, wherein the patch backing layer has a first polymer composition, wherein the overlay backing layer has a second polymer composition, wherein the first polymer composition and second polymer composition comprise substantially the same polymers, and wherein both the first polymer composition and the second polymer composition comprise one or more synthetic polymers selected from the group consisting of polyolefin oils, polyester, polyethylene, polyvinylidine, chloride, and polyurethane.

14. The transdermal drug delivery patch with integrated overlay claim 12, wherein the first elastomer, the second elastomer, or both the first elastomer and the second elastomer are selected from the group consisting of polyacrylates, polyisobutylene (PIB), natural rubber, silicone, and styrene rubber.

15. The transdermal drug delivery patch with integrated overlay of claim 14, wherein the first elastomer is a polyacrylate comprising hydroxyl functional groups, and wherein the second elastomer is a polyacrylate comprising hydroxyl functional groups, and wherein the overlay adhesive layer further comprises a tackifying resin.

16. The transdermal drug delivery patch with integrated overlay of claim 12, wherein:
(i) the transdermal delivery patch is a drug-in adhesive transdermal patch, and wherein the patch adhesive layer comprises a therapeutically effective amount of a drug; or
(ii) the transdermal drug delivery patch is a reservoir-type transdermal patch comprising a drug reservoir, and wherein the drug reservoir comprises a therapeutically effective amount of a drug; and
wherein the drug is selected from the group consisting of clonidine, scopolamine, oxybutynin, lesopritron, estradiol, levonorgestrel, fentanyl, albuterol, labetalol, atropine, haloperidol, isosorbide dinitrate, nitroglycerin, norethindrone acetate, nicotine, benztropine, secoverine, dexsecoverin, arecoline, buprenorphine, donepezil hydrochloride, donepezil base, lidocaine, selegiline, rivastigmine, methylphenidate, diclofenac, ondansetron, varencicline, oxymorphone, rotigotine, and granisetron.

17. The transdermal drug delivery patch with integrated overlay of claim 12, wherein the transdermal drug delivery patch with integrated overlay has a peel adhesion that is at least 1.5-fold greater as compared to the transdermal drug delivery patch and wherein the overlay adhesive layer has a thickness of at least 1 mil.

18. A method of transdermally administering a therapeutically effective amount of a drug to an individual in need of such drug, the method comprising:
(a) providing the transdermal drug delivery patch with integrated overlay of claim 12; and
(b) applying to intact skin of the individual the transdermal drug delivery patch with integrated overlay for at least about 2 days.

19. The method of claim 18, wherein the transdermal drug delivery patch with integrated overlay is applied to the intact skin of the individual for at least about 4 days.

20. The method of claim 19, wherein the transdermal drug delivery patch with integrated overlay is applied to the intact skin of the individual for at least about 7 days.
